(19) United States
(12) Patent Application Publication
(10) Pub. No.: US 2023/0050696 A1
(43) Pub. Date: Feb. 16, 2023

(54) Exercise Bike and Handlebar Assembly

(71) Applicant: Suijimanbu (Shanghai) Sports Technology Co., Ltd., Shanghai (CN)

(72) Inventor: Cheng Chen, Shanghai (CN)

(73) Assignee: Suijimanbu (Shanghai) Sports Technology Co., Ltd., Shanghai (CN)

(21) Appl. No.: 17/733,308

(22) Filed: Apr. 29, 2022

(30) Foreign Application Priority Data
Aug. 13, 2021 (CN) 202110930530.6
Jan. 20, 2022 (CN) 202220153077.2

(51) Int. Cl. A63B 21/00; A63B 22/06; A63B 71/06; A63B 24/00; G16H 20/30

(52) U.S. Cl. CPC A63B 21/4035; A63B 22/0605; A63B 71/0622; A63B 24/0075; G16H 20/30; A63B 2071/0658; A63B 2071/0625; A63B 2024/0012; A63B 2071/0638; A63B 2024/0068

(57) ABSTRACT

An exercise bike and a handlebar assembly are provided. The handlebar assembly includes: a rod-shaped handlebar; a support post; a first connecting component comprising a first recess, and further comprising a first tenon and a second tenon; a second connecting component comprising a first side, a second side away from the first side and a sidewall connecting the first side to the second side, wherein the second side of the second connecting component is provided with a second recess, and further provided with a first mortise and a second mortise. When the second recess is aligned with the first recess, a first through-hole is formed for the handlebar passing through, the first tenon is inserted in the first mortise, and the second tenon is inserted in the second mortise. The handlebar assembly can be easily assembled to the exercise bike.

### CROSS-REFERENCE TO RELATED APPLICATIONS

This application claims priority to Chinese Patent Application No. 202110930530.6, filed on Aug. 13, 2021, and Chinese Patent Application No. 202220153077.2, filed on Jan. 20, 2022, the entire contents of inch are incorporated herein by reference.

### TECHNICAL FIELD

The present disclosure relates to exercise equipment technology, particularly an exercise bike and a handlebar assembly.

### BACKGROUND

Exercise with an exercise bike can effectively burn calories and has low exercise skill requirements. Therefore, the exercise bike has become one of people's favorite exercise equipment. The handlebar assembly is usually separated from the bike frame during storage and transportation, to decrease the occupied space of the exercise bike. However, the traditional handlebar assembly has holes at the bottom portion to avoid influence on the appearance. The user needs to attach the handlebar to the exercise bike by screwing the bolts from the back of the handlebar assembly, which is very inconvenient.

Therefore, there is a need in the art for the handlebar assembly that can be conveniently assembled to the exercise bike.

### SUMMARY

In one aspect of the present disclosure, a handlebar assembly is provided, including: a rod-shaped handlebar; a support post; a first connecting component comprising a first recess, and further comprising a first tenon and a second tenon located at two sides of the first recess; a second connecting component comprising a first side, a second side away from the first side and a sidewall connecting the first side to the second side, wherein the first side of the second connecting component is sleeved on the support post, the second side of the second connecting component is provided with a second recess, and further provided with a first mortise and a second mortise located at two sides of the second recess; wherein, when the second recess is aligned with the first recess, a first through-hole is formed for the handlebar passing through, the first tenon is inserted in the first mortise, and the second tenon is inserted in the second mortise.

In one aspect of the present disclosure, an exercise bike is provided, including: a bike frame; a saddle connected to the bike frame; a drive assembly connected to the bike frame; at least one wheel connected to the drive assembly; a pedal assembly connected to the drive assembly, wherein the pedal assembly drives the at least one wheel to rotate through the drive assembly; and the handlebar assembly above, wherein the handlebar assembly is connected to the bike frame through the support post.

### BRIEF DESCRIPTION OF THE DRAWINGS

The foregoing and other features, and advantages of the invention are apparent from the following detailed description taken in conjunction with the accompanying drawings in which:

FIG. 1 is a perspective view of a handlebar assembly according to an embodiment of the present disclosure;

FIG. 2 is an exploded view of the handlebar assembly according to the embodiment of the present disclosure;

FIG. 3 is an enlarged view of area A in FIG. 2;

FIG. 4 is another exploded view of the handlebar assembly according to the embodiment of the present disclosure;

FIG. 5 is an enlarged view of area B in FIG. 4;

FIG. 6 is a cross-sectional view of the handlebar assembly according to the embodiment of the present disclosure;

FIG. 7 is a perspective view of an exercise bike according to an embodiment of the present disclosure;

FIG. 8 is a side view of the exercise bike according to the embodiment of the present disclosure;

FIG. 9 is a flow chart of an exercise method according to the embodiment of the present disclosure;

FIG. 10 is a schematic view of a display interface of a display and computing device according to the embodiment of the present disclosure;

FIG. 11 is a flow chart of generating a first exercise guiding video according to the embodiment of the present disclosure;

FIG. 12 is a flow chart of generating a movement instruction sequence according to the embodiment of the present disclosure;

FIG. 13 is a flow chart of generating a second exercise guiding video according to the embodiment of the present disclosure;

FIG. 14 is a flow chart of generating a CGA including special-effect/animated feedbacks according to the embodiment of the present disclosure;

FIG. 15 is a flow chart of providing interactive feedback according to the embodiment of the present disclosure;

FIG. 16 is a flow chart of displaying a leaderboard display area according to an embodiment of the present disclosure;

FIG. 17 is a schematic view of a display interface including the leaderboard display area on a display and computing device according to an embodiment of the present disclosure;

FIG. 18 is a block diagram of an exercise server according to an embodiment of the present disclosure.

### DETAILED DESCRIPTION

In the following, embodiments of the present disclosure will be described in detail with reference to the figures. The concept of the present disclosure can be implemented in a plurality of forms, and should not be understood to be limited to the embodiments described hereafter. On the contrary, these embodiments are provided to make the present disclosure more comprehensive and understandable, and so the conception of the embodiments can be fully conveyed to those skilled in the art. Same reference signs in the figures refer to same or similar elements, so a repeated description of them will be omitted.

Besides, the technical features, assemblies, and characteristics can be combined in any appropriate way in one or more embodiments. In the following, more specific details are provided to give a full understanding of the embodiments of the present disclosure. However, those skilled in the art should realize that the technical proposal can also be realized without one or more of the specific details, or with other assemblies or components. In other conditions, some common assemblies or components well known in the art are not described to avoid making the present disclosure unclear. Some blocks in the block diagram represent functional entities and maybe not in correspondence to physical or logical individual entities. The functional entities can be realized using software, or using one or more hardware modules or integrated modules, or using different networks and/or processors and/or micro control devices.

The terms “one”, “the”, “at least one” refer to one or more elements, components, etc. The terms “comprise”, “include” and “have” means that other elements or components may exist except for listed ones.

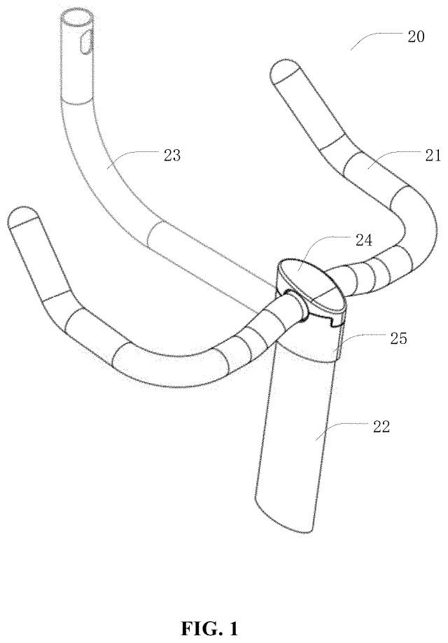

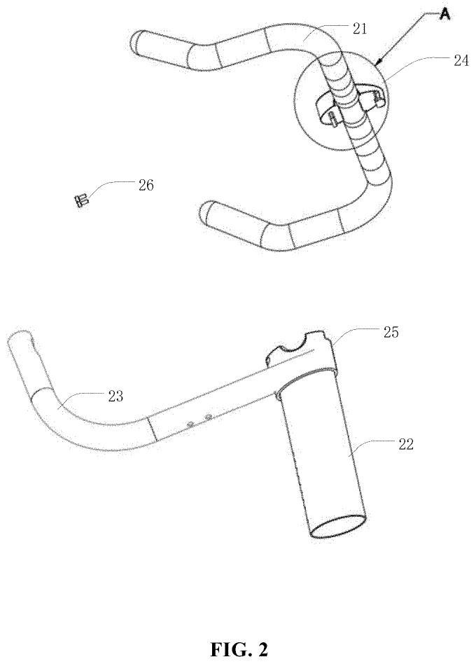

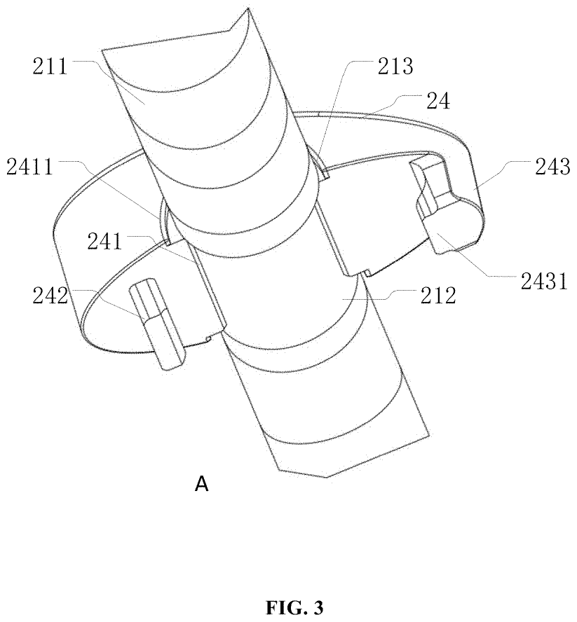

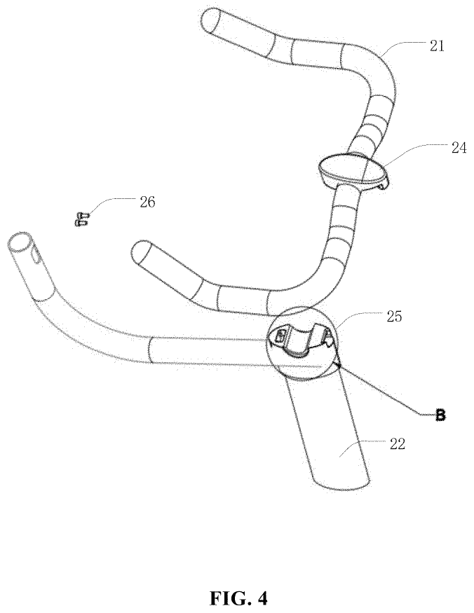

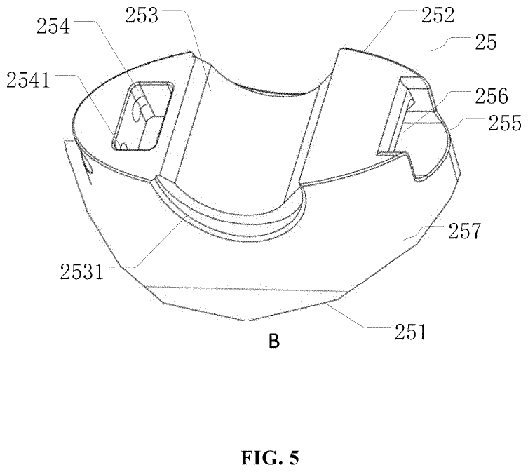

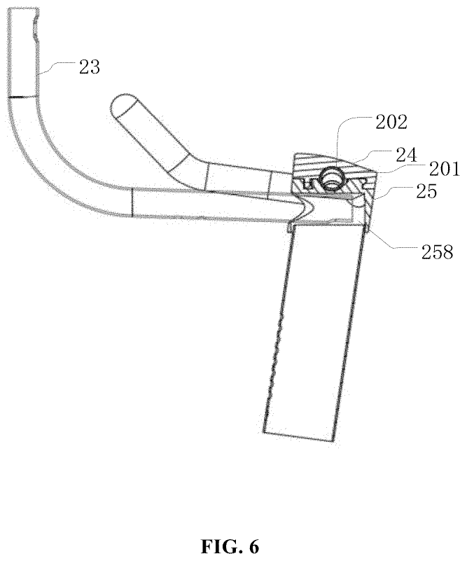

The handlebar assembly of the present disclosure will be described in detail, referring to FIGS. 1-6. FIG. 1 is a perspective view of a handlebar assembly according to an embodiment of the present disclosure. FIG. 2 is an exploded view of the handlebar assembly according to the embodiment of the present disclosure. FIG. 3 is an enlarged view of area A in FIG. 2. FIG. 4 is another exploded view of the handlebar assembly according to the embodiment of the present disclosure. FIG. 5 is an enlarged view of area B in FIG. 4. FIG. 6 is a cross-sectional view of the handlebar assembly according to the embodiment of the present disclosure.

The handlebar assembly 20 includes a rod-shaped handlebar 21, a supporting post 22, a first connecting component 24, and a second connecting component 25.

The first connecting component 24 includes a first recess 241, and further includes a first tenon 242 and a second tenon 243 located at two sides of the first recess 241.

The second connecting component 25 includes a first side 251, a second side 252 away from the first side 251, and a sidewall 257 connecting the first side 251 to the second side 252. The first side 251 of the second connecting component 25 is sleeved on the support post 22. The second side 252 of the second connecting component 25 is provided with a second recess 253, and is further provided with a first mortise 254 and a second mortise 255 located at two sides of the second recess 253.

When the second recess 253 is aligned with the first recess 241, a first through-hole 201 is formed for the handlebar passing through. The first tenon 242 is inserted in the first mortise 254, and the second tenon 243 is inserted in the second mortise 55.

Therefore, the first connecting component 24 and the second connecting component 25 are connected through two pairs of tenon and mortise structures, the first through-hole is located between the two pairs of tenon and mortise structures. The connecting strength between the rod-shaped handlebar 21 and the support post 22 is improved.

The installation of the handlebar assembly 20 is described here in detail. Firstly, the handlebar 21 is inserted in the first recess 241 of the first connecting component 24, and the second connecting component 25 is sleeved on the support post 25. Then the first connecting component 24, on which the handlebar 21 is mounted, is connected to the second connecting component 25 in a direction from top to bottom shown in FIG. 2 and FIG. 4, by inserting the first tenon 242 and the second tenon 243 into the first mortise 254 and the second mortise 255. At this time, a part of the handlebar 21 protruding from the first recess 241 is inserted in the second recess 253 of the second connecting component 25, thereby finishing the installation of the handlebar assembly. Since the handlebar assembly 20 can be assembled in the direction from top to bottom, the user can easily and conveniently assemble the handlebar assembly 20 to an exercise bike without squatting or turning over the exercise bike.

In some embodiments, a gap exists between the first tenon 242 and an edge of the first connecting component 24. That is, the first tenon 242 doesn't extend to the edge of the first connecting component 24. Correspondingly, a gap exists between the first mortise 254 and an edge of the second side 252. That is, the first mortise 254 doesn't extend to the edge of the second side 252. Therefore, the first tenon 242 only has one degree of motion freedom in one direction (the up and down direction shown in FIG. 6) relative to the first mortise 254, to improve the connecting strength between the first tenon 242 and the first mortise 254.

In some embodiments, the second tenon 243 extends from the edge of the first connecting component 24 in a direction opposite to a concave direction of the first recess 241. The opening of the second mortise 255 faces the second surface 252 and the sidewall 257 of the second connecting component 25. Therefore, the second tenon 243 can be inserted in the second mortise 255 from the sidewall 257 of the second connecting component 25. Furthermore, an end of the second tenon 243 away from the first recess 241 is provided with a tongue-shaped portion 2431 extending towards the first mortise 254. Correspondingly, an inner wall of the second mortise 255 has a tongue-shaped recess 256 extending towards the first mortise 254. When the second tenon 243 is inserted in the second mortise 254, the tongue-shaped portion 2431 is received in the tongue-shaped recess 256. Therefore, when the second tenon 243 is inserted in the second mortise 255 from the sidewall 257 of the second connecting component 25, a degree of motion freedom in the up and down direction shown in FIG. 6 of the second tenon 243 relative to the second mortise 255 is limited by the tongue-shaped portion 2431 and the tongue-shaped recess 256. Therefore, after the first tenon 242 is connected to the first mortise 254, and the second tenon 243 is connected to the second mortise 255, the degree of motion freedom, in the up and down direction shown in FIG. 6, of the first tenon 242 relative to the first mortise 254 is limited by the tongue-shaped portion 2431 and the tongue-shaped recess 256. At the same time, a degree of motion freedom, in the left and right direction shown in FIG. 6, of the second tenon 243 relative to the second mortise 255 is limited by the first tenon 242 and the first mortise 254. Therefore, after the first connecting component 24 is connected to the second connecting component 25, the degrees of motion freedom, in the up and down direction, in the left and right direction, and in the front and rear direction, are all limited. Therefore, the first connecting component 24 is stably connected to the second connecting component 25. Furthermore, a gap exists between the first tenon 242 and the first mortise 254 after the first tenon 242 is inserted in the first mortise 254. Therefore, when the handlebar assembly is disassembled, the first connecting component 24 can be moved towards a right side shown in FIG. 6, to form a gap between the second tenon 243 and the inner wall of the second mortise 255, to provide a space for the tilt of the first connecting component 24. After the first connecting component 24 is tilted, the first tenon 242 can be moved away from the first mortise 254, and the second tenon 243 can be moved away from the second mortise 255, to finish the disassembly of the handlebar assembly.

In some embodiments, the sidewall 257 of the second connecting component 25 is provided with a threaded hole 2541 connected with the first mortise 254. One side of the first tenon 242 away from the second tenon 243 is provided with a threaded hole corresponding to the threaded hole 2541. The handlebar assembly 20 further includes a threaded bolt 26. The threaded bolt 26 is screwed in the threaded hole 2541 on the sidewall 257 and the threaded hole on the first tenon 242, to secure the first tenon 242 to the first mortise 254. Therefore, the connection strength is improved between the first connecting component 24 and the second connecting component 25.

In some embodiments, the handlebar 21 includes a handlebar portion 211, a connecting portion 212, and a step portion 213 connecting the handlebar portion 211 to the connecting portion 212. An outer diameter of the connecting portion 212 is smaller than an outer diameter of the handlebar portion 211, to form the step portion 213 at the connection position between the handlebar portion 211 and the connecting portion 212. The connecting portion 212 is received in the through-hole 201, and the step portion 213 contacts against two ends of the through-hole 201, to prevent the handlebar 21 from sliding in the through-hole 201. Furthermore, an end of the first recess 241, defined in the axial direction, is provided with a first step recess 2411, an end of the second recess 253 in the axial direction is provided with a second step recess 2531. The first step recess 2411 is aligned with the second step recess 253 to form a step through-hole 202 for the step portion 213 passing through. Therefore, since the step portion 213 can be received in the first step recess 2411 and the second step recess 2531, there is no need for forming the step portion 213 to adapt to the shape of the sidewall of the first connecting component 24 and the sidewall of the second connecting component 25, thereby the manufacturing process of the handlebar 21 is simplified. Furthermore, the step portion 213 will be concealed to provide a clean appearance at the connection position between the handlebar 21 and the first connecting component 24 and the connection position between the handlebar 21 and the second connecting component 25.

In some embodiments, the sidewall 257 of the second connecting component 25 is provided with a second through-hole 258 for an accessory connecting rod 23 passing through. The accessory connecting rod 23 is used for connecting to other accessories.

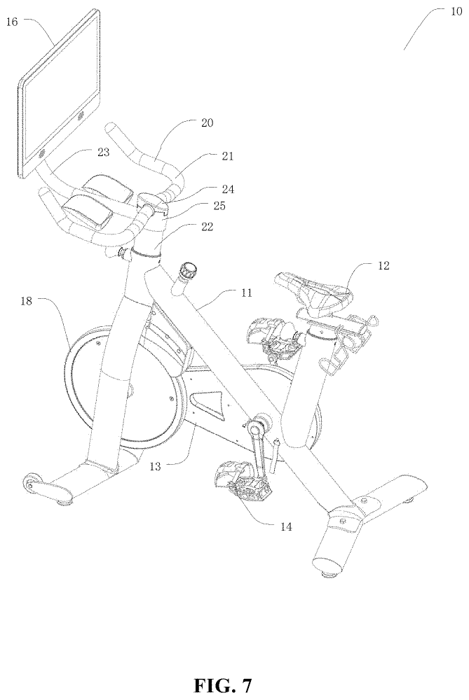

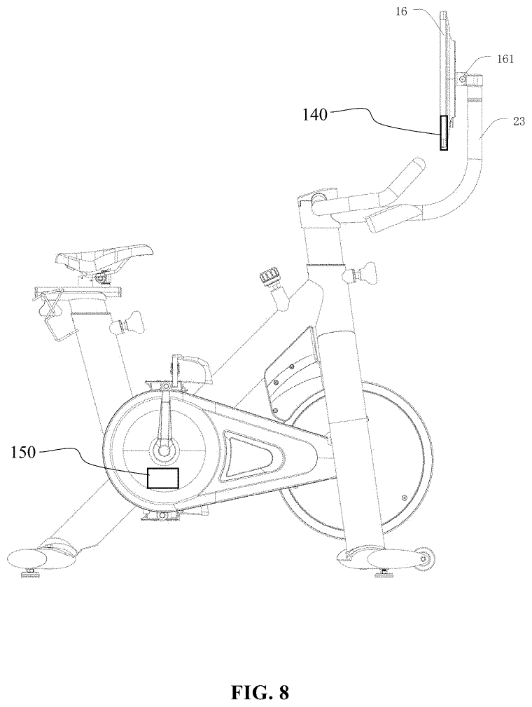

The present disclosure further provides an exercise bike, which will be described in detail by combining FIGS. 7 and 8. FIG. 7 is a perspective view of an exercise bike according to an embodiment of the present disclosure. FIG. 8 is a side view of the exercise bike according to the embodiment of the present disclosure.

The exercise bike 10 includes a bike frame 11, a saddle 12, a drive assembly 13, at least one wheel 18, a pedal assembly 14 and a handlebar assembly 20.

The bike frame 11 can be in a shape of “Z”. A bottom portion of the bike frame 11 is provided with support posts, of which the height can be adjusted. The bottom portion of the bike frame 11 is further provided with rollers. In some embodiments, the height of the support posts can be adjusted to leave a gap between the rollers and the ground, thereby preventing the movement of the exercise bike 10 when the user is exercising. When the exercise bike is needed to move, the height of the support posts can be adjusted to make the roller contact with the ground, such that the exercise bike can be moved to a destination with the assistance of the rollers.

The saddle 12 can be connected to the bike frame 11 through a lifting assembly. The lifting assembly may include a fixed column, a movable column sleeved on the fixed column, and a limit component used to limit the movable column's movement. The saddle 12 is connected to the movable column, such that the lifting assembly can adjust height of the saddle 12.

The drive assembly 13 is connected to the bike frame 11.

The exercise bike may include one or two wheels. In the embodiment, there are two wheels 18 connected to a drive side and a follower side of the drive assembly 13, respectively. For example, the rear wheel is connected to the drive side of the drive assembly, while the front wheel is connected to the follower side of the drive assembly.

The pedal assembly 14 is connected to the drive assembly 13. The pedal assembly 14 may include a pair of pedals and a pair of rotating rods. Each pedal is connected to the drive assembly 13 through each rotating rod. The rotating rods can be connected to the central shaft of the rear wheel. When the user applies force to the pedals, the pedals rotate together with the rotating rods about the central shaft of the rear wheel, to rotate the rear wheel. At the same time, the rear wheel drives the front wheel to rotate through the drive assembly 13.

The handlebar assembly 20 is connected to the bike frame 11 through the support post 22. The height of the support post 22 relative to the bike frame 11 can be adjusted.

In some embodiments, the exercise bike 10 may further includes a display and computing device 16. The display and computing device 16 is connected to the accessory connecting rod 23 of the handlebar assembly 20 through a rotating component 162. The rotating component 162 can rotate relative to the accessory connecting rod 23 to rotate the display and computing device 16. The display and computing device 16 is configured to configured to play videos and audios, connected to outer sensors and receiving sensing data from the outer sensors.

The above description of the exercise bike and the handlebar assembly of the present disclosure teaches by way of example and not by limitation. Therefore, the matter contained in the above description or shown in the accompanying drawings should be interpreted as illustrative and not in a limiting sense. The features of different embodiments can be applied independently or combined, which are all included in the protection scope of the present disclosure.

The exercise bike and the handlebar assembly of the present disclosure at least have the following advantages.

The rod-shaped handlebar can be assembled to the support post through the first connecting component and the second connecting component. When the handlebar assembly is assembled, the user only needs to connect the first connecting component to the rod-shaped handle, connect the second connecting component to the support post, and insert the first tenon and the second tenon of the first connecting component into the first mortise and the second mortise of the second connecting component. Therefore, the assembly is very convenient for the user. Furthermore, the tenons of the first connecting component both face the second connecting component to provide a clean appearance of the top surface of the first connecting component. Therefore, the present disclosure can provide a clean appearance of the handlebar assembly, and make it easier to assemble the handlebar assembly to the exercise bike.

In the embodiment, the display and computing device 16 can be a display screen facing the bike frame 11 and configured to play videos and audios, and process programs and algorithms. In some other embodiments, the display and computing device 16 can also be a projector facing away from the bike frame 11 and configured to play videos and audios, and process programs and algorithms. Wherein the display and computing device 16 provides a user interface, so that a user can operate content displayed on the display and computing device 16 by voice, touch, gesture, etc. The user operation may include selecting music, selecting videos, selecting exercise classes, adjusting volume, etc.

The exercise bike further includes a plurality of bike sensor devices 150 and a control device 140. The bike sensor devices 140 are configured to track and collect user performance data. FIG. 8 only schematically illustrates a position of the bike sensor devices 150. In other embodiments, the bike sensor devices 150 can be provided on any one or more of the pedals 14, the drive assembly 13, and the wheels 18, to track and collect the cadence, resistance, etc. Therefore, the tracked and collected data can be user performance data. Furthermore, the user performance data can further include heart rate. For example, the bike sensor devices 150 can further include a sensor on the handlebar 21 for sensing heart rate. In some alternative embodiments, an intelligent wearable apparatus such as an intelligent bracelet can be used for sensing the heart rate of the user. In some alternative embodiments, the bike sensor devices 150 can further include a pressure sensor on the saddle 12 for sensing whether the user is on or out of the bike saddle 12.

In the embodiment, the control device 140 can be integrated to the display and computing device 16, or the control device 140 can be an individual device independent of the display and computing device 16 and mounted at any position of the bike frame 11. The control device 140 can communicate with the display and computing device 16 using wired or wireless connections.

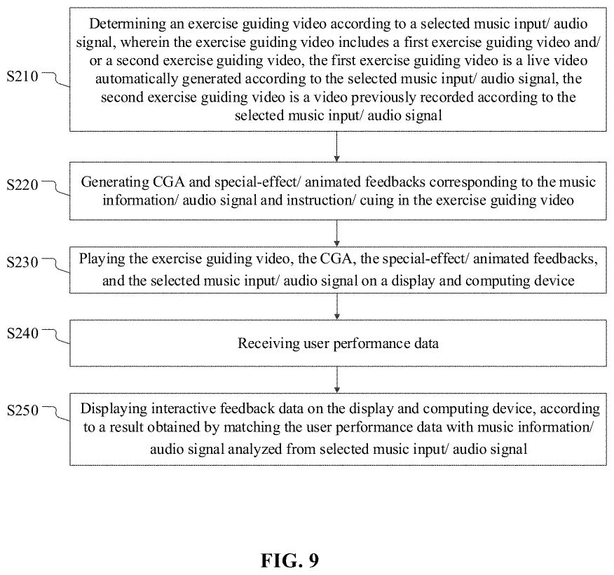

The control device is configured to: receive an exercise guiding video determined according to a selected music input/audio signal, wherein the exercise guiding video comprises a first exercise guiding video and/or a second exercise guiding video, the first exercise guiding video is a live video generated automatically according to the selected music input/audio signal, the second exercise guiding video is a video previously recorded according to the selected music input/audio signal; receive CGA and special-effect/animated feedbacks; control the display and computing device to display the exercise guiding video, the CGA, the special-effect/animated feedbacks and the selected music input/audio signal; receive the user performance data from the bike sensor devices; receive interactive feedback data generated or updated according to a result obtained by matching the user performance data with music information/audio signal analyzed from selected music input/audio signal; control the display and computing device to display the interactive feedback data. The details of the above steps will be further described in the following by combining FIGS. 9-17. Wherein, the CGA at least includes stage and/or background.

In the embodiment, the control device 140 can receive the live or previously recorded exercise guiding video from a remote exercise server. The server is configured to provide the exercise guiding video, calculate the interactive feedback data, and match other data, etc. Therefore, the hardware requirement of the control device of the exercise equipment can be lowered down by applying the complex data and algorithm processing to the server, so that the hardware of the exercise equipment can be simplified. In some alternative embodiments, the control device of the exercise equipment can execute a part of the data processing and calculation, to avoid data delay caused by communication problems. The server can be in the form of a server cluster or a distributed server.

In the embodiment, during exercise, the user can select the exercise guiding video or receive the exercise guiding video recommended by the server. The music input/audio signal, the exercise guiding video, the variable CGA, and special-effect/animated feedbacks are overlaid and integrated when displayed on the display and computing device 16. The user can see the guidance in the exercise guiding video, hear the music input/audio signal, and exercise on the excise bike. The bike sensor devices 150 provide the user performance data to the control device 140 and/or the server. The control device 140 and/or the server can provide the interactive feedback data to the display and computing device 16 according to the matching and analyzing result of the user performance data and the music input/audio signal, so that the display and computing device 16 can show the interactive feedback data to the user.

Therefore, in the present disclosure, on the one hand, individualized service is provided to the user by providing multi-layer video including a variable CGA, special-effect/animated feedbacks, and the interactive feedback data, so that the user can experience an immersive reality extended reality during exercise. On the other hand, by playing the music input/audio signal, and generating or previously recording the exercise guiding video according to the music input/audio signal, the exercise movements are tightly combined with the music information/audio signal of the music input/audio signal to increase entertainment benefit during exercise, so that the user is easier to develop an exercise habit. Furthermore, compared to matching and analyzing the exercise movements to the movements in the exercise guiding video, matching and analyzing the user performance data to the music information/audio signal of the music input/audio signal has a faster data processing speed and a faster feedback speed.

The control device 140 is configured to store executable instructions, when the control device 140 execute the instructions, an exercise method is performed.

FIG. 9 is a flow chart of an exercise method according to an embodiment of the present disclosure. As shown in FIG. 9, the exercise method includes the following steps S210-S250.

S210: determining an exercise guiding video according to a selected music input/audio signal, wherein the exercise guiding video includes a first exercise guiding video and/or a second exercise guiding video, the first exercise guiding video is a live video automatically generated according to the selected music input/audio signal, the second exercise guiding video is a video previously recorded according to the selected music input/audio signal.

In the embodiment, the music input/audio signal is selected according to a user selection. For example, the user can select a music file in a provided music library as the selected music input audio signal. For another example, the user can upload a local music file as the selected music input/audio signal. For another example, the user can upload a hyperlink of a third-party music file, from which the server can obtain the music input/audio signal and related information. In another embodiment, the music input/audio signal can be streaming media data.

In the embodiment, the music information/audio signal is stored in the music library with a mapping relationship to the music files in the music library. The local music file uploaded by the user can be analyzed by the server/control device to extract the music information/audio signal thereof. The music information/audio signal of the selected music input/audio signal includes music attributes/features and a timeseries/sequence with signals of rhythmic events/features. The timeseries/sequence with signals of rhythmic events/features can include a plurality of segments with signals of rhythmic events/features. The timeseries/sequence with signals of rhythmic events/features can further include bpm (beats per minute). Each segment with signals of rhythmic events/features may further include a timing and location of each beat in the segment with signals of rhythmic events/features of the music input/audio signal and duration of each segment with signals of rhythmic events/features. In some embodiments, each segment with signals of rhythmic events/features can include eight beats. In some alternative embodiments, a number of beats per minute in each segment with signals of rhythmic events/features can be different. The timeseries/sequence with signals of rhythmic events/features may further include a downbeat time series including a timing and location of each downbeat in the music input/audio signal. The music attributes/features include a variety of measurements or quantification of music energy, the music attributes/features further include one or more of music duration, music segments, lyrics, genre, and artist. In the embodiment, the music input/audio signal can be separated into a plurality of music segments according to wording, sentences or segments of the lyrics, the separating information is the information of the music segments. A variety of measurements or quantification of music energy can be varying measurements or quantification of audio intensity between different segments with signals of rhythmic events/features or between different music segments. In some alternative embodiments, the music attributes/features can further include other kinds of characteristics of the music input/audio signal.

In another embodiment, the music input/audio signal is selected by matching and analyzing the music information/audio signal of the music input/audio signal to a persona and user behavior pattern. The persona and user behavior pattern can be obtained by learning from basic data and/or exercise class data of the user. The basic data of the user can include height, age, gender, weight, etc., of the user. The exercise class data of the user may include a class level, movement preference, aesthetic style preference, etc. The movement preference can be obtained by learning from a number of each movement performed by the user, completion status of each movement, and/or other movement data. The aesthetic style preference of the user can be obtained by learning from a number of using each CGA, a number of using each special effect, feedback data after playing the CGA and the special-effect/animated feedbacks in the exercise class data. Furthermore, a matching and analyzing model can be used to obtain a matching and analyzing relationship between the music information/audio signal of the music input/audio signal and the persona and user behavior pattern. In other embodiments, the matching and analyzing can be realized in other ways. For example, a plurality of preferred music files can be obtained as the persona and user behavior pattern from a music playlist of the user in music applications, a number of playing each music file, or other information. Then the music file is selected by matching and analyzing the plurality of preferred music files to the music files in the music library.

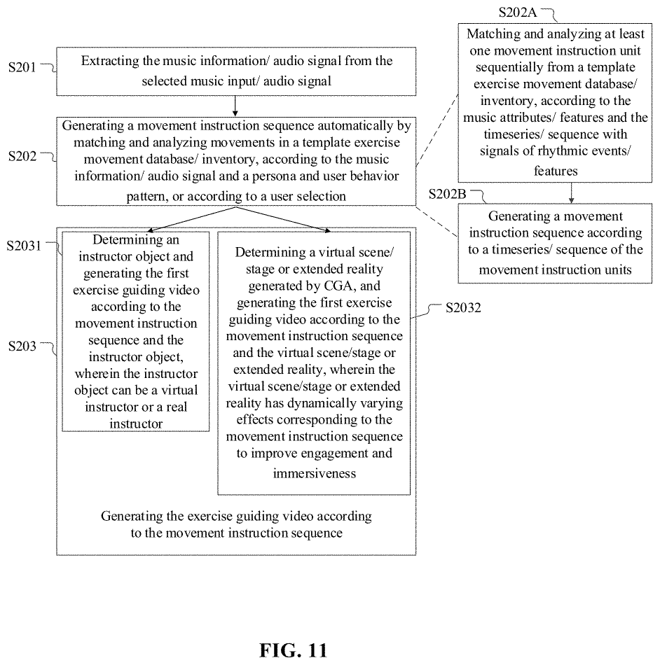

The process of generating and previously recording the exercise guiding video will be described in detail by combining FIGS. 11-13 in the following.

S220: generating CGA and special-effect/animated feedbacks corresponding to the music information/audio signal and instruction/cuing in the exercise guiding video.

In the embodiment, the CGA is used for a background of the exercise guiding video. The CGA can be a static image or a dynamic animation. The CGA can represent a virtual scene/stage or extended reality. For example, the extended reality can be a sea scene, a forest scene, a city scene, or a stage, etc. The virtual scene can be a sea scene, a forest scene, a city scene, or a stage, etc., built of a plurality of elements. In other embodiments, the CGA can also be a background having solid color background or alphabet-inspired background.

In the embodiment, the special-effect/animated feedbacks can be virtual light effects overlaid and integrated on the CGA. The special-effect/animated feedbacks can also be special processing effects to elements in the CGA (for example, image scaling, making the element move/feedback in synchronization with the beats/rhythm, etc.).

In some embodiments, the CGA and the special-effect/animated feedbacks can be matched according to the persona and user behavior pattern. For example, the CGA and the special-effect/animated feedbacks are matched and analyzed to the user's aesthetic style preference in the persona and user behavior pattern. In some other embodiments, the CGA and the special-effect/animated feedbacks are matched according to the music information/audio signal. For example, the CGA and the special-effect/animated feedbacks are matched and analyzed to the music segments, lyrics, genre. In an embodiment, the CGA and the special-effect/animated feedbacks can be labeled, and a model including mapping relations between the music information/audio signal and the labels is previously built. Then the matching and analyzing between the CGA and the special-effect/animated feedbacks and the music information/audio signal can be realized using the model. In another embodiment, the CGA and the special-effect/animated feedbacks can be matched and analyzed to the persona and user behavior pattern and the music information/audio signal. In the embodiment, a first score is obtained by matching and analyzing the CGA and the special-effect/animated feedbacks to the persona and user behavior pattern, and a second score is obtained by matching and analyzing the CGA and the special-effect/animated feedbacks to the music information/audio signal. A total score is obtained by weighted summation of the first score and the second score, and the CGA and the special-effect/animated feedbacks are selected according to the total score.

S230: playing the exercise guiding video, the CGA, the special-effect/animated feedbacks, and the selected music input/audio signal on a display and computing device.

In the embodiment, the step of playing the exercise guiding video, the CGA, the special-effect/animated feedback, and the selected music input/audio signal on a display and computing device further includes synthesizing the exercise guiding video, the CGA, the special-effect/animated feedbacks, and the selected music input/audio signal. Therefore the display and computing device can play an integrated video and audio file.

S240: receiving user performance data.

In the embodiment, the user performance data can be received from different sensor devices of different exercise devices when different exercise devices are used for exercise. For example, when the exercise bike is used for exercise, the user performance data can be received from the bike sensor devices of the exercise bike, and the user performance data can include cadence, resistance, whether the user is on or out of the bike saddle, and heart rate, etc. tracked and collected by the bike sensor devices. When the exercise accessory is used for exercise, the user performance data can be received from the accessory sensor devices of the exercise accessory, and the user performance data can include angular rates, linear velocity, position and heart rate, etc. tracked and collected by the accessory sensor devices. When the user exercises without any exercise device, the user video stream can be received from the video capturing device mounted on the display and computing device, and the user performance data can be identified from the user video stream. The user performance data can include angular rates, linear velocity and position etc., of body parts identified from the user video stream. Identifying the movements of the user from the video stream can be realized by identifying the skeleton points, skeleton feature vectors, angles between the skeleton feature vectors, etc. In different embodiments, one of the above exercise modes can be used independently, or a combination of two or more of the above exercise modes can be used.

S250: displaying interactive feedback data on the display and computing device, according to a result obtained by matching the user performance data with music information/audio signal analyzed from selected music input/audio signal.

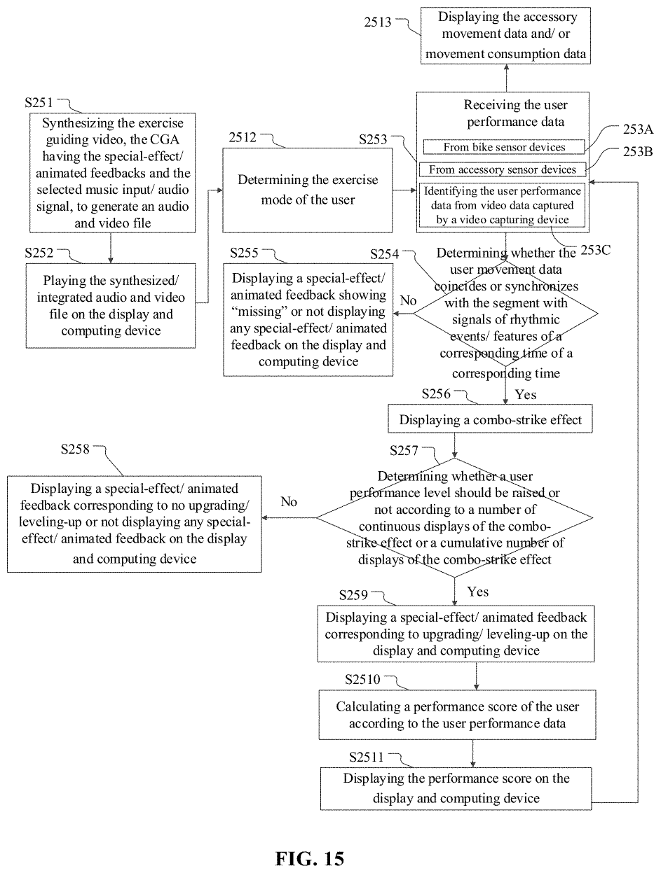

In the embodiment, the interactive feedback data provided can include whether the user movements match to the beat or other audio signals, whether combo-strike is achieved (determined according to the matching result of the user movements with the beat or other audio signals), a number of combo-strikes, a user performance level, a user performance score, user exercise data, etc. The interactive feedback data will be described in detail by combining FIGS. 15-17.

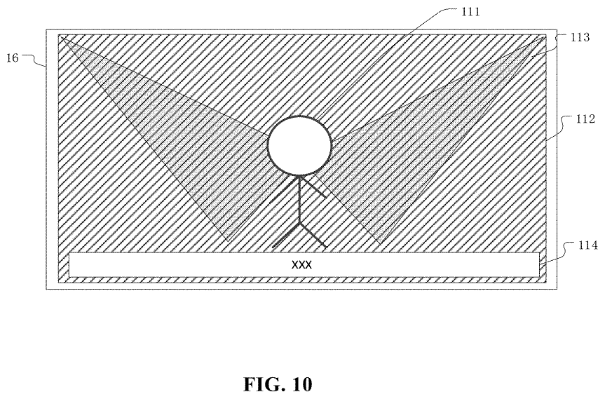

FIG. 10 is a schematic view of a display interface of a display and computing device according to an embodiment of the present disclosure. As shown in FIG. 10, the interface displayed by the display and computing device 16 includes CGA 112, special-effect/animated feedbacks 113, exercise guiding video 111 including an instructor object, and an interactive feedback area 114. FIG. 10 only schematically illustrates a kind of interface provided in the present disclosure. In other embodiments, the interface can be different from that shown in FIG. 10.

In the exercise method of the present disclosure, live/streamed videos with multiple layers of visual effects for guiding the user exercise can be provided to the user by playing the exercise guiding video, the CGA, the special-effect/animated feedbacks, and the interactive feedback data in an integrated/multi-layered way. By generating the exercise guiding video according to the music input/audio signal, and generating the interactive feedback data according to the matching and analyzing result between the music file and the user performance data, the exercise process of the user can be guided by the music input/audio signal, the entertainment benefit and the interactive experience during the user exercise are improved.

FIG. 11 is a flow chart of generating a first exercise guiding video according to an embodiment of the present disclosure. As shown in FIG. 11, the first exercise guiding video is generated by the following steps.

S201: extracting the music information/audio signal from the selected music input/audio signal.

In the embodiment, the music information/audio signal can include a timeseries/sequence with signals of rhythmic events/features. In some embodiments, the timeseries/sequence with signals of rhythmic events/features can be extracted by a trained model. In another embodiment, the timeseries/sequence with signals of rhythmic events/features can be extracted by processing the audio data of the selected music input/audio signal. In the embodiment, the timeseries/sequence with signals of rhythmic events/features can be obtained by: identifying the beats from the selected music input/audio signal, obtaining the timing and location of each beat in the selected music input/audio signal, separating the beats of the selected music input/audio signal into a plurality of segments with signals of rhythmic events/features, and sequencing the plurality of segments with signals of rhythmic events/features by time to get the timeseries/sequence with signals of rhythmic events/features. Furthermore, bpm (beats per minute) can also be calculated according to the number of beats per minute identified in the selected music input/audio signal.

In the embodiment, the music information/audio signal of the selected music input/audio signal can include music attributes/features. The music attributes/features can include music duration, lyrics, genre, and artist, etc. The music duration, lyrics, genre, and artist can be stored with a mapping relationship to the selected music input/audio signal; therefore, the music information/audio signal can be obtained directly according to the selected music input/audio signal. The music attributes/features can include music segments, wherein the separating information is the information of the music segments. The variety of measurements or quantification of music energy can be varying measurements or quantification of audio intensity between different segments with signals of rhythmic events/features or between different music segments. Therefore, a variety of measurements or quantification of music energy can be obtained by processing the audio signal of the selected music input/audio signal.

S202: generating a movement instruction sequence automatically by matching and analyzing movements in a template exercise movement database/inventory, according to the music information/audio signal and a persona and user behavior pattern, or according to a user selection.

In the embodiment, the template exercise movement database/inventory includes a plurality of movement instruction units. The movement data can be stored in the template exercise movement database/inventory according to the movement instruction units. The movement data can include a two- or three-dimensional movement model/mechanism. For example, in a movement model/mechanism, the skeleton points, skeleton feature vectors, angles between the skeleton feature vectors, etc., are stored as objects of the movement instruction units. Position, moving track, moving speed of the objects of the movement instruction units are stored as the movement attributes/features of the objects of the movement instruction units.

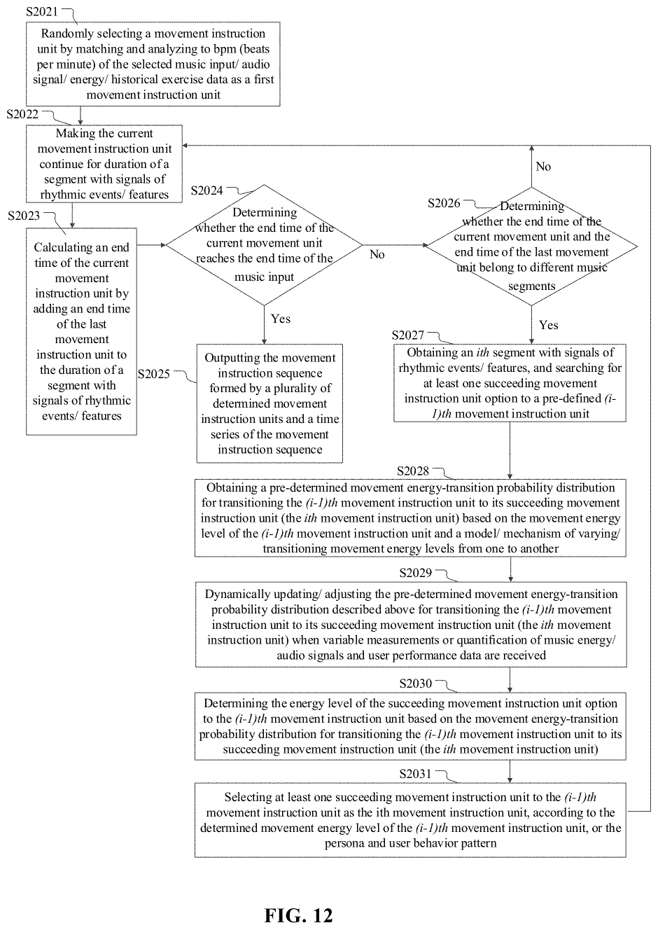

In the embodiment, step S202 can further include: step S202A: matching and analyzing at least one movement instruction unit sequentially from a template exercise movement database/inventory, according to the music attributes (such as beats per minutes, musical structure, music energy, rhythmic segmentation, etc.)/features and the timeseries/sequence with signals of rhythmic events/features, wherein the template exercise movement database/inventory includes a plurality of movement instruction units; and step S202B: generating a movement instruction sequence according to a timeseries/sequence of the movement instruction units. In the embodiment, the details of step S202 will be described in the following by combining FIG. 12.

S203: generating the exercise guiding video according to the movement instruction sequence.

In the embodiment, the exercise guiding video generated in S203 is the first exercise guiding video. Step S203 can include step S2031: determining an instructor object and generating the first exercise guiding video according to the movement instruction sequence and the instructor object, wherein the instructor object can be a virtual instructor or a real instructor. In the embodiment, the virtual instructor can be a virtual instructor figure or an animated figure. The virtual instructor can be stored together with mapping relationships to figure data configured for building movements. The figure data can include virtual figure display data (for example, muscles, skins, etc.) based on the skeleton points, skeleton feature vectors, angles between the skeleton feature vectors, etc. Therefore, the virtual figure display data can be generated by matching and analyzing the data of each movement instruction unit in the movement instruction sequence to the stored virtual figure display data and synthesizing the data of each movement instruction unit with the matched virtual figure display data. In the embodiments, the real instructor can record content videos of the movement instruction units according to the template exercise movement database/inventory. Therefore, the first exercise guiding video can be generated by matching and analyzing the movement instruction sequence to the content video of each movement instruction unit previously recorded by the selected real instructor.

Furthermore, the instructor object can be determined according to a user selection. In other embodiments, the instructor object can also be determined according to the music information/audio signal of the music input/audio signal and/or the persona and user behavior pattern. For example, user-preferred instructor objects can be determined according to historical exercise class data of the user. For another example, a user-preferred label of the instructor object can be determined according to the historical exercise class data of the user, and the instructor object can be determined by matching and analyzing the user-preferred label of the instructor object to the stored labels of the instructor objects. For another embodiment, a model can be used to learn the relationships between the music information/audio signal of the music input/audio signals and the instructor objects, to realize the matching and analyzing between the music information/audio signal of the music input/audio signals and the instructor objects by the model. Wherein the music information/audio signal of the music input/audio signals can include only a part of the music attributes/features, for example, the genre, artist, lyrics, etc., to increase the efficiency of training and using the model. For another example, the instructor object can be determined by matching and analyzing the instructor objects to the music information/audio signal and the persona and user behavior pattern.

Step S203 can further include step 2032: determining a virtual scene/stage or extended reality generated by CGA, and generating the first exercise guiding video according to the movement instruction sequence and the virtual scene/stage or extended reality, wherein the virtual scene/stage or extended reality has dynamically varying effects corresponding to the movement instruction sequence to improve engagement and immersiveness. In the embodiment, the virtual scene/stage or extended reality generated by CGA uses a scene or stage to show the movement instruction sequence, which is different from the aforementioned front layer using a form of an instructor object showing the movement instruction sequence. The virtual scene/stage or extended reality can be in the form of characters, graphics, etc., and the virtual scene/stage or extended reality has dynamically varying effects corresponding to the movement instruction sequence to improve engagement and immersiveness to show the movement instruction sequence.

Furthermore, the virtual scene/stage or extended reality generated by CGA can be selected by the user. In other embodiments, the virtual scene/stage or extended reality generated by CGA can also be determined according to the music information/audio signal of the music input/audio signal and/or the persona and user behavior pattern. For example, user-preferred virtual scene/stage or extended reality generated by CGA can be determined according to the historical exercise class data of the user. For another example, a user-preferred label of the virtual scene/stage or extended reality generated by CGA can be determined according to the historical exercise class data of the user, and the virtual scene/stage or extended reality generated by CGA can be determined by matching and analyzing the user-preferred label of the virtual scene/stage or extended reality generated by CGA to the stored labels of the virtual scenes/stages or extended reality generated by CGA. For another embodiment, a model can be used to learn the mapping relationships between the music information/audio signal of the music input/audio signals and the virtual scene/stage or extended reality generated by CGA, to realize the matching and analyzing between the music information/audio signal of the music input/audio signals and the virtual scene/stage or extended reality by the model. Wherein the music information/audio signal of the music input/audio signals can include only a part of the music attributes/features, for example, the genre, artist, lyrics, etc., to increase the efficiency of training and using the model. For another example, the virtual scene/stage or extended reality generated by CGA can be determined by matching and analyzing the virtual scenes/stages or extended reality generated by CGA to the music information/audio signal and the persona and user behavior pattern.

In the embodiment, while generating the first exercise guiding video according to the movement instruction sequence, a preset rule can be used to adjust the video to make the transition between the movement instruction units smoother.

FIG. 12 is a flow chart of generating the movement instruction sequence. As shown in FIG. 12, the movement instruction sequence is generated by the following steps:

S2021: randomly selecting a movement instruction unit by matching and analyzing to bpm (beats per minute) of the selected music input/audio signal/energy/historical exercise data as a first movement instruction unit.

In the embodiment, each movement instruction unit can be stored with a mapping relationship to the corresponding bpm (beats per minute).

S2022: making the current movement instruction unit continue for duration of a segment with signals of rhythmic events/features.

For example, a movement duration of the current movement instruction unit is two beats, the duration of a segment with signals of rhythmic events/features is eight beats. So, the current movement instruction unit is repeated four times to continue for the duration of a segment with signals of rhythmic events/features.

S2023: calculating an end time of the current movement instruction unit by adding an end time of the last movement instruction unit to the duration of a segment with signals of rhythmic events/features.

S2024: determining whether the end time of the current movement instruction unit reaches the end time of the music input/audio signal.

If the end time of the current movement instruction unit reaches the end time of the music input/audio signal, the matching and analyzing of all the segments with signals of rhythmic events/features of the selected music input/audio signal have been completed, then step S2025 is executed, outputting the movement instruction sequence formed by a plurality of determined movement instruction units and a time series of the movement instruction sequence.

If the end time of the current movement instruction unit hasn't reached the total duration of the music input/audio signal, step S2026 is executed, determining whether the end time of the current movement instruction unit and the end time of the last movement instruction unit belong to different music segments.

If the end time of the current movement instruction unit and the end time of the last movement instruction unit belong to different music segments, step S2022 is executed again.

In the embodiment, step S2026 can be omitted according to different exercise requirements and exercise movements. For example, the number of exercise movements using the exercise bike is less than other exercise modes, each movement instruction unit is made to continue for the duration of each music segment. When the music segment changes, steps S2027 to S2031 are executed to determine the subsequent movement instruction unit again. For another example, the number of exercise movements using the exercise accessory is more than other exercise modes, step S2026 can be omitted, to make the movement instruction unit only continue for the duration of a segment with signals of rhythmic events/features.

After step 2026, if the end time of the current movement instruction unit and the end time of the last movement instruction unit belong to a same music segment, then step S2027 is executed, obtaining an ith segment with signals of rhythmic events/features, and searching for at least one succeeding movement instruction unit option to a pre-defined (i−l)th movement instruction unit.

In the embodiment, i is an integer ranging from 2 to N, and N is a number of the segments with signals of rhythmic events/features in the timeseries/sequence with signals of rhythmic events/features. Wherein an initial value of i is 2, and every time the following step S2031 is executed, i=i+1.

In an embodiment, transition problems exist between different movement instruction units. Therefore, each movement instruction unit is related to a plurality of succeeding movement instruction unit options.

S2028: obtaining a pre-determined movement energy-transition probability distribution for transitioning the (i−l)th movement instruction unit to its succeeding movement instruction unit (the ith movement instruction unit) based on the movement energy level of the (i−l)th movement instruction unit and a model/mechanism of varying/transitioning movement energy levels from one to another.

In the embodiment, the movement energy level of each movement instruction unit is a preset movement intensity. A high-intensity movement instruction unit succeeding to another high-intensity movement instruction unit may cause excessive exercise intensity for the user, and may cause sports injuries to the user. A low-intensity movement instruction unit succeeding to another low-intensity movement instruction unit may cause insufficient movement intensity for the user, and expected exercise effects could not be achieved. In the embodiment, the model/mechanism of varying/transitioning movement energy levels from one to another can be obtained by learning the energy level varying measurements or quantification between the movement instruction units from historical exercise data. For example, the historical exercise data can be historical exercise class data. Sample data can be obtained by separating the movement instruction units in the historical exercise class data and determining the energy levels of the movement instruction units in the historical exercise class data. Then the model/mechanism of varying/transitioning movement energy levels from one to another can be trained using the sample data. The model/mechanism of varying/transitioning movement energy levels from one to another can provide a basic and general method of varying/transitioning movement energy levels from one to another. In step S2028, the movement energy level of the (i−l)th movement instruction unit can be input to the model/mechanism of varying/transitioning movement energy levels from one to another to obtain the pre-determined movement energy-transition probability distribution for transitioning the (i−l)th movement instruction unit to its succeeding movement instruction unit (the ith movement instruction unit). For example, the probability distribution of the movement energy level of transitioning the (i−l)th movement instruction unit to a first movement instruction unit option is a %, the probability distribution of the movement energy level of transitioning the (i-1)th movement instruction unit to a second movement instruction unit option is b %, and the probability distribution of the movement energy level of transitioning the (i−l)th movement instruction unit to a third movement instruction unit option is c %.

S2029: dynamically updating/adjusting the pre-determined movement energy-transition probability distribution described above for transitioning the (i−l)th movement instruction unit to its succeeding movement instruction unit (the ith movement instruction unit) when variable measurements or quantification of music energy/audio signals and user performance data are received.

In the embodiment, by step S2029, the pre-determined movement energy-transition probability distribution described above for transitioning the (i−l)th movement instruction unit to its succeeding movement instruction unit (the ith movement instruction unit) are further updated/adjusted according to a variety of measurements or quantification of music energy and the user performance data, based on the basic and general movement energy-transition probability distribution provided by the model/mechanism of varying/transitioning movement energy levels from one to another.

In the embodiment, the user performance data can include user live performance data or user performance data in a recent time period. Therefore, the user performance data can be used for determining whether the user can adapt to the model/mechanism of varying/transitioning movement energy levels from one to another. If yes, there is no need to adjust the obtained pre-determined movement energy-transition probability distribution. If no, it is determined that whether the user can complete the movement easily (for example, the user has a low heart rate during exercise) or the user feels hard to complete the movement (for example, the user has a high heart rate during exercise). If the user can complete the movement easily, probabilities of high energy levels can be raised and probabilities of low energy levels can be decreased in the movement energy-transition probability distribution. If the user feels hard to complete the movement, the probabilities of high energy levels can be decreased and the probabilities of low energy levels can be raised in the movement energy-transition probability distribution.

In the embodiment, a variety of measurements or quantification of music energy can be used for representing the varying measurements or quantification of the audio intensity. In general, when the audio intensity of the music input/audio signal is higher, the energy level of the current movement is higher; when the audio intensity of the music input/audio signal is lower, the energy level of the current movement is lower. Therefore, the music input/audio signal and the movements can be tightly combined. In the embodiment, if an energy level of the current music segment/segment with signals of rhythmic events/features is higher than an energy level of the last music segment/segment with signals of rhythmic events/features, probabilities of energy levels of the succeeding movement instruction unit option to the last movement instruction unit higher than the energy level of the last movement instruction unit can be raised, probabilities of energy levels of the succeeding movement instruction unit option to the last movement instruction unit lower than the energy level of the last movement instruction unit can be decreased. If the energy level of the current music segment/segment with signals of rhythmic events/features is lower than the energy level of the last music segment/segment with signals of rhythmic events/features, the probabilities of energy levels of the succeeding movement instruction unit option to the last movement instruction unit higher than the energy level of the last movement instruction unit can be decreased, probabilities of energy levels of the succeeding movement instruction unit option to the last movement instruction unit lower than the energy level of the last movement instruction unit can be raised. If the energy level of the current music segment/segment with signals of rhythmic events/features is equal to the energy level of the last music segment/segment with signals of rhythmic events/features, there is no need to adjust the pre-determined movement energy-transition probability distribution.

S2030: determining the energy level of the succeeding movement instruction unit option to the (i−l)th movement instruction unit based on the movement energy-transition probability distribution for transitioning the (i−l)th movement instruction unit to its succeeding movement instruction unit (the ith movement instruction unit).

In the embodiment, an energy level having the highest probability can be determined to be the movement energy level of the succeeding movement instruction unit option to the (i−l)th movement instruction unit.

S2031: selecting at least one succeeding movement instruction unit to the (i−l)th movement instruction unit as the ith movement instruction unit, according to the determined movement energy level of the (i−l)th movement instruction unit, or the persona and user behavior pattern.

In some embodiments, a movement instruction unit can be determined as the ith movement instruction unit, by selecting from at least one succeeding movement instruction unit option of the (i−l)th movement instruction unit according to the determined movement energy level. In other embodiments, the movement instruction unit can be selected by the user as the ith movement instruction unit, from at least one succeeding movement instruction unit option of the (i−l)th movement instruction unit according to the determined movement energy level. In other embodiments, the movement instruction unit can be determined as the ith movement instruction unit, by selecting from at least one succeeding movement instruction unit option of the (i−l)th movement instruction unit according to the determined movement energy level and the persona and user behavior pattern. Wherein the persona and user behavior pattern includes the user's preferred movements, which can be stored in the form of a preferred movement set. Therefore, the ith movement instruction unit can be determined by matching and analyzing the preferred movement set with at least one succeeding movement instruction unit.

After step S2031 is executed, step S2022 is executed again.

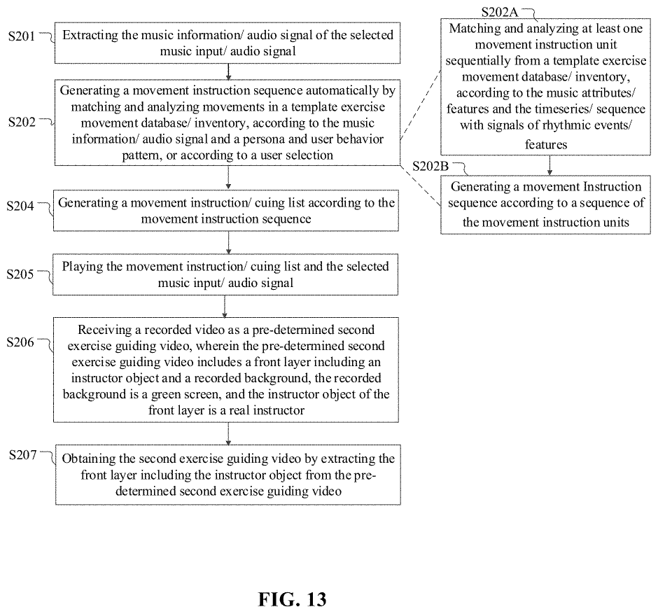

FIG. 13 is a flow chart of generating a second exercise guiding video according to an embodiment of the present disclosure. As shown in FIG. 13, the second exercise guiding video is generated by the following steps.

S201: extracting the music information/audio signal of the selected music input/audio signal.

S202: generating a movement instruction sequence automatically by matching and analyzing movements in a template exercise movement database/inventory, according to the music information/audio signal and a persona and user behavior pattern, or according to a user selection.

In the embodiment, step S202 can further include: step S202A: matching and analyzing at least one movement instruction unit sequentially from a template exercise movement database/inventory, according to the music attributes/features and the timeseries/sequence with signals of rhythmic events/features; and step S202B: generating a movement instruction sequence according to a sequence of the movement instruction units, the details of step S202 will be referred to the aforementioned description by combining FIG. 12.

S204: generating a movement instruction/cuing list according to the movement instruction sequence.

In the embodiment, the movement instruction/cuing list is used to show the movement instruction sequence to be recorded. In some embodiments, the movement instruction/cuing list can be the first exercise guiding video generated by the steps shown in FIG. 11. In other embodiments, the movement instruction/cuing list can show the movement data (stored in the template exercise movement database/inventory) of each movement instruction unit of the movement instruction sequence. In some other embodiments, the movement instruction/cuing list can show a cue in text form.

S205: playing the movement instruction/cuing list and the selected music input/audio signal.

In the embodiment, the movement instruction/cuing list and the selected music input/audio signal are synchronized in a time sequence. Therefore, the movement instruction/cuing list and the selected music input/audio signal synchronized in time sequence/timeseries can be played for the instructor so that the instructor can record a pre-determined second exercise guiding video under the guidance of the movement instruction/cuing list and the selected music input/audio signal. Furthermore, the time sequence of the music cue file can be set ahead of the selected music input/audio signal for a preset time. The instructor has enough time to understand the movement cue after seeing the movement cue video. Therefore, the movements performed by the instructor according to the movement instruction/cuing list can be synchronized with the selected music input/audio signal in the time sequence.

S206: receiving a recorded video as a pre-determined second exercise guiding video, wherein the pre-determined second exercise guiding video includes a front layer including an instructor object and a recorded background, the recorded background is a green screen, and the instructor object of the front layer is a real instructor.

S207: obtaining the second exercise guiding video by extracting the front layer including the instructor object from the pre-determined second exercise guiding video.

In the embodiment, the second exercise guiding video is recorded using a green screen to facilitate the removal of the green screen. Therefore, the green screen can be easily removed from the pre-determined second exercise guiding video to extract the front layer including the instructor object, to generate the second exercise guiding video.

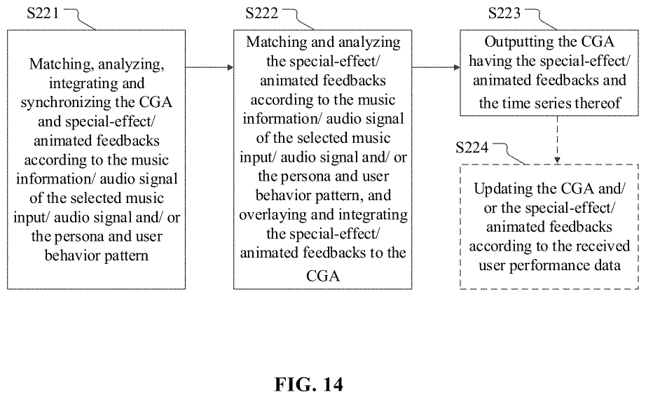

FIG. 14 is a flow chart of generating a CGA including special-effect/animated feedbacks according to an embodiment of the present disclosure. As shown in FIG. 14, the CGA is by the following steps:

S221: matching, analyzing, integrating and synchronizing the CGA and special-effect/animated feedbacks according to the music information/audio signal of the selected music input/audio signal and/or the persona and user behavior pattern.

In the embodiment, the CGA can be selected from a CGA library, according to one or more of the music genres, the aesthetic style preference (preferred CGA style) in the persona and user behavior pattern, the style requirement to the CGA of a class/community marketing activity. In the embodiment, each CGA is stored in the CGA library with a mapping relationship to a style label. Therefore, the CGA can be selected and determined by matching and analyzing the style label.

S222: matching and analyzing the special-effect/animated feedbacks according to the music information/audio signal of the selected music input/audio signal and/or the persona and user behavior pattern, and overlaying and integrating the special-effect/animated feedbacks to the CGA.

For example, the special-effect/animated feedbacks can be light effects, particle effects, etc. In the embodiment, the special-effect/animated feedbacks can be selected from a special-effect/animated library, according to the aesthetic style preference (preferred CGA style) of the user and/or the music genre. Furthermore, the varying of the special-effect/animated feedback can be determined according to the timeseries/sequence with signals of rhythmic events/features of the music input/audio signal (including a beat time series and a downbeat time series), music segments, a variety of measurements or quantification of music energy. For example, the light effects can flash following the beats in the beat time series. The brightness of the light effect can be increased at the timing and location of the downbeat in the downbeat time series. The brightness can vary following the varying measurements or quantification of the music energy. For example, when the music energy of the current music segment/segment with signals of rhythmic events/features is greater, the brightness of the light effect is greater; when the music energy of the current music segment/segment with signals of rhythmic events/features is smaller, the brightness of the light effect is smaller.

Therefore, the determined special-effect/animated feedbacks and the method of changing the special-effect/animated feedbacks can be overlaid and integrated to the CGA.

S223: outputting the CGA having the special-effect/animated feedbacks and the time series thereof.

S224: updating the CGA and/or the special-effect/animated feedbacks according to the received user performance data.

In some embodiments, step S224 can be omitted. Therefore, the CGA and the special-effect/animated feedbacks obtained from step S223 can be output. In other embodiments, step S224 is executed to improve the interactive experience of the user. For example, when the user performance data shows that the current movement intensity is excessive for the user, the CGA and/or the special-effect/animated feedbacks can be adjusted to more smoothing CGA and/or special-effect/animated feedbacks, to help the user to alleviate exercise fatigue. For example, when the user performance data shows that the user is not exerting full effort during current exercise, the CGA and/or the special-effect/animated feedbacks can be adjusted to more striking CGA and/or special-effect/animated feedbacks to urge the user to exercise.

FIG. 15 is a flow chart of providing interactive feedback according to an embodiment of the present disclosure. As shown in FIG. 15, the interactive feedback is provided by the following steps.

S251: synthesizing the exercise guiding video, the CGA having the special-effect/animated feedbacks and the selected music input/audio signal, to generate an audio and video file.

S252: playing the synthesized/integrated audio and video file on the display and computing device.

S2512: determining the exercise mode of the user.

In an embodiment only having one exercise mode, step S2512 can be omitted.

In the embodiment, the exercise modes include an exercise bike mode, an exercise accessory mode, and a computer vision mode.

S253: receiving the user performance data.

In the embodiment, if the user exercise mode is the exercise bike mode, step S253A is executed, receiving the user performance data from bike sensor devices of an exercise bike. If the user exercise mode is the exercise accessory mode, step S253B is executed, receiving the user performance data from accessory sensor devices of an exercise accessory. If the user exercise mode is the computer vision mode, step S253C is executed, receiving video stream of the user movements from a video capturing device; and identifying the user performance data from the video stream.

S254: determining whether the user performance data coincides or synchronizes with the segment with signals of rhythmic events/features of a corresponding time.

If the user performance data doesn't coincide or synchronize with the segment with signals of rhythmic events/features of a corresponding time, step S255 is executed, displaying a special-effect/animated feedback showing “missing” or not displaying any special-effect/animated feedback on the display and computing device.

If the user performance data coincides or synchronizes with the segment with signals of rhythmic events/features of a corresponding time, step S256 is executed, displaying a combo-strike effect.

S257: determining whether a user performance level should be raised or not according to a number of continuous displays of the combo-strike effect or a cumulative number of displays of the combo-strike effect.

If the user performance level shouldn't be raised, step 5258 is executed, displaying a special-effect/animated feedback corresponding to no upgrading/leveling-up or not displaying any special-effect/animated feedback on the display and computing device.

If the user performance level should be upgraded, step 5259 is executed, displaying a special-effect/animated feedback corresponding to upgrading/leveling-up on the display and computing device.

In the embodiment, steps S257-S259 are executed to inspire the user by displaying the special-effect/animated feedback corresponding to upgrading/leveling-up. The user performance level can represent the current exercise amount/movement intensity. The user performance level can be obtained by calculating a number of continuous displays of the combo-strike effect or a cumulative number of displays of the combo-strike effect. For example, when the number of continuous displays of the combo-strike effect is greater than a preset threshold, the user performance level should be upgraded. For another example, when the cumulative number of displays of the combo-strike effect is greater than a preset threshold, the user performance level should be upgraded/leveled up. When the cumulative number of displays of the combo-strike effect minus the cumulative number of displays of the combo-strike effect before the last time upgrade is greater than a preset threshold, the user performance level should be upgraded. Other varying modes can also be used in other embodiments of the present disclosure. In some embodiments, steps S257-S259 can also be omitted.

S2510: calculating a performance score of the user according to the user performance data.

In the embodiment, in step S2510, a unit score of the current movement instruction unit performed by the user can be firstly calculated, then the performance score of the user can be obtained by accumulating the unit score of the previous movement instruction units.

In an embodiment using the exercise bike, the unit score can be calculated based on the resistance of the user performance data.

In the embodiment, when step S2510 is executed, the current movement instruction unit of the user is matched and analyzed to the music information/audio signal of the music input/audio signal. That is to say, when step S2510 is executed, the current movement instruction unit is completed by the user, and a basic unit score can be obtained. The unit performance score can be calculated based on the basic unit score and the resistance of the user performance data. For example, a weight coefficient is calculated according to the resistance of the user performance data, and the unit score can be obtained by multiplying the weight coefficient and the basic unit score. The weight coefficient is positively related to the resistance of the user performance data. In other embodiments, the performance score can be obtained in other ways.

S2511: displaying the performance score on the display and computing device.

Wherein, after step S253, step S2513 is executed, displaying the accessory movement data and/or movement consumption data. The movement consumption data is obtained by at least calculating based on the accessory movement data. The accessory movement data includes one or more of heart rate, movement duration, movement intensity.

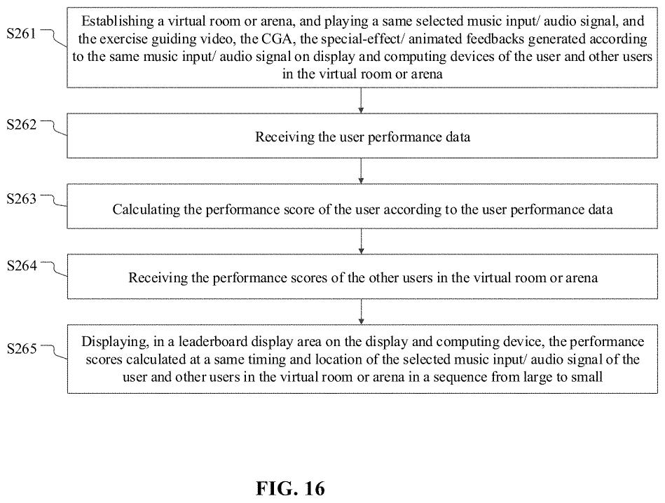

FIG. 16 is a flow chart of displaying a leaderboard display area according to an embodiment of the present disclosure. As shown in FIG. 16, the leaderboard display area is displayed by the following steps.

S261: establishing a virtual room or arena, and playing a same selected music input/audio signal, and the exercise guiding video, the CGA, the special-effect/animated feedbacks generated according to the same music input/audio signal on display and computing devices of the user and other users in the virtual room or arena.

In the embodiment, a user can send, via the exercise device (or a mobile device associated with the exercise device), an invitation of establishing a virtual room or arena to the exercise devices (or mobile devices associated with the exercise devices) of other users. When at least one user receives the invitation and sends feedback data, the communication channel between the users in the virtual room or arena is built.

In an alternative embodiment, in the virtual room or arena, the display and computing devices of the users play the same content. In some embodiments, in the virtual room or arena, the display and computing devices of the user and other users in the virtual room or arena play the same selected music input/audio signal, and the exercise guiding video, the CGA, the special-effect/animated feedbacks generated according to the same music input/audio signal.

S262: receiving the user performance data.

S263: calculating the performance score of the user according to the user performance data.

S264: receiving the performance scores of the other users in the virtual room or arena.

S265: displaying, in a leaderboard display area on the display and computing device, the performance scores calculated at a same timing and location of the selected music input/audio signal of the user and other users in the virtual room or arena in a sequence from large to small. Wherein the displayed performance scores include the performance scores of the other users in the same virtual room or arena with the user at a same timing and location of the selected music input/audio signal.

In the embodiments, the display and computing devices of the other users in a same virtual room or arena play a same selected music input/audio signal at the same time with the display and computing device of the user. The live performance scores of the other users in the same virtual room or arena can be received, so that the displayed performance scores include the performance scores of the other users in the same virtual room or arena with the user at a same timing and location of the selected music input/audio signal.

In another embodiment, the display and computing devices of the other users in the same virtual room or arena don't have to play the same selected music input/audio signal at the same time with the display and computing device of the user. Displaying the performance scores calculated at a same timing and location of the selected music input/audio signal of the user and other users in the virtual room or arena can be realized by receiving the performance scores of the other users calculated at the current timing and location of the selected music input/audio signal played by the user. In other words, the performance scores of the user at various timing and locations of the selected music input/audio signal can be stored. Therefore, other users can receive the scores for display.

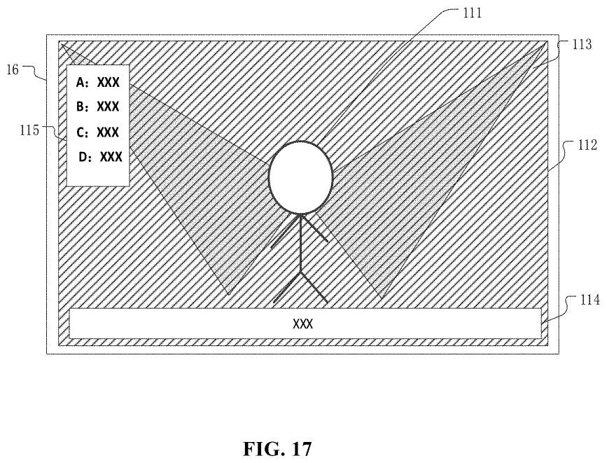

As shown in FIG. 17, the display and computing device 16 includes CGA 112, special-effect/animated feedbacks 113, exercise guiding video 111 including an instructor object, an interactive feedback area 114, and a leaderboard display area 115. The leaderboard display area 115 can show user accounts and/or avatars of the users, and corresponding performance scores. The sequence of the performance scores displayed in the leaderboard display area 115 dynamically varies following the varying of the performance scores. FIG. 17 only schematically illustrates a kind of display interface provided by the present disclosure. In other embodiments, the display interface can be different from that shown in FIG. 17.

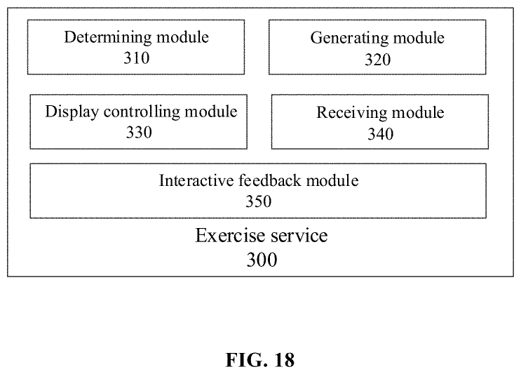

FIG. 18 is a block diagram of an exercise server according to an embodiment of the present disclosure. The server 300 can communicate and interact with the exercise equipment shown in FIGS. 1-8, to provide related video and data service. The server 300 includes a determining module 310, a generating module 320, a display controlling module 330, a receiving module 340, and an interactive feedback module 350.

The determining module 310 is configured to determine an exercise guiding video according to a selected music input/audio signal, wherein the exercise guiding video includes a first exercise guiding video and/or a second exercise guiding video, the first exercise guiding video is a live video automatically generated according to the selected music input/audio signal, the second exercise guiding video is a video previously recorded according to the selected music input/audio signal. The generating module 320 is configured to generate CGA (Computer Generated Animation) and special-effect/animated feedbacks corresponding to the music information/audio signal and instruction/cuing in the exercise guiding video. The display controlling module 330 is configured to play the exercise guiding video, the CGA, the special-effect/animated feedbacks, and the selected music input/audio signal on a display and computing device. The receiving module is configured to receive user performance data. The interactive display module 350 is configured to display interactive feedback data on the display and computing device, according to a result obtained by matching the user performance data with music information/audio signal analyzed from selected music input/audio signal.

FIG. 18 only schematically illustrative the exercise server 300 provided by the present disclosure. In other embodiments, the modules in the server 300 can be separated or combined, or other modules can be added to the server 300. The server 300 can be composed of software, hardware, firmware, plug-in components, or any combination thereof

Compared to the existing technology, the exercise bike with the above control device has the following advantages.

During exercise, live/streamed videos with multiple layers of visual effects for guiding the user exercise can be provided to the user by playing the exercise guiding video, the CGA, the special-effect/animated feedbacks, and the interactive feedback data in an integrated/multi-layered way. By generating the exercise guiding video according to the music input/audio signal, and generating the interactive feedback data according to the matching and analyzing result between the music file and the user performance data, the exercise process of the user can be guided by the music input/audio signal, the entertainment benefit and the interactive experience during the user exercise are improved.

All relative and directional references (including up, down, upper, lower, top, bottom, side, front, rear, left, right, and so forth) are given by way of example to aid the reader's understanding of the examples described herein. They should not be read to be requirements or limitations, particularly as to the position, orientation, or use unless specifically set forth in the claims. It should be noted that, if the devices in the figures are flipped upside down, the component described as “above” will become the component as “below”. When a structure is “on” another structure, it may mean that the structure is integrally formed on the said another structure, or that the structure is “directly” disposed on the said another structure, or that the structure is “indirectly” disposed on the said another structure through a further another structure. Connection references (e.g., attached, coupled, connected, joined, and the like) are to be construed broadly and may include intermediate members between a connection of elements and relative movement between elements. As such, connection references do not necessarily infer those two elements are directly connected and in fixed relation to each other, unless specifically set forth in the claims.

In the description above, the terms “one embodiment”, “some embodiments”, “example” etc. are used to describe different embodiments. Furthermore, the technical features in different embodiments can be combined in adequate ways to form new embodiments, which are all included in the present disclosure.

The above is a detailed description of the present disclosure in connection with the specific preferred embodiments, and the specific embodiments of the present disclosure are not limited to the description. Modifications and substitutions can be made without departing from the spirit and scope of the present disclosure.

What is claimed is:

1. A handlebar assembly comprising:
a rod-shaped handlebar;
a support post;
a first connecting component comprising a first recess, and further comprising a first tenon and a second tenon located at two sides of the first recess;
a second connecting component comprising a first side, a second side away from the first side, and a sidewall connecting the first side to the second side, wherein the first side of the second connecting component is sleeved on the support post, the second side of the second connecting component is provided with a second recess, and further provided with a first mortise and a second mortise located at two sides of the second recess;
wherein, when the second recess is aligned with the first recess, a first through-hole is formed for the handlebar passing through, the first tenon is inserted in the first mortise, and the second tenon is inserted in the second mortise.

2. The handlebar assembly of claim 1, wherein, a gap exists between the first tenon and an edge of the first connecting component; a gap exists between the first mortise and an edge of the second side.

3. The handlebar assembly of claim 1, wherein, the second tenon extends from an edge of the first connecting component in a direction opposite to a concave direction of the first recess, an opening of the second mortise faces the second side and the sidewall of the second connecting component.

4. The handlebar assembly of claim 3, wherein, an end of the second tenon away from the first recess is provided with a tongue-shaped portion extending towards the first tenon, an inner wall of the second mortise is provided with a tongue-shaped recess extending towards the first recess, the tongue-shaped portion is received in the tongue-shaped recess when the second tenon is inserted in the second mortise.

5. The handlebar assembly of claim 1, wherein, the sidewall of the second connecting component is provided with a threaded hole connected with the first mortise, the handlebar assembly further comprises a threaded bolt screwed in the threaded hole to fix the first tenon in the first mortise.

6. The handlebar assembly of claim 1, wherein, the handlebar comprises a handlebar portion, a connecting portion and a step portion connecting the handlebar portion to the connecting portion, an outer diameter of the connecting portion is smaller than an outer diameter of the handlebar portion, and the connecting portion is received in the first through-hole.

7. The handlebar assembly of claim 6, wherein, a first step recess is provided on an end of the first recess defined in an axial direction, a second step recess is provided on an end part of the second recess defined in the axial direction, the first step recess is aligned with the second step recess to form a step through-hole for the step portion passing through.

8. The handlebar assembly of claim 1, wherein, the sidewall of the second connecting component is provided with a second through-hole for an accessory connecting rod passing through.

9. An exercise bike comprising:
a bike frame;
a saddle connected to the bike frame;
a drive assembly connected to the bike frame;
at least one wheel connected to the drive assembly;
a pedal assembly connected to the drive assembly, wherein the pedal assembly drives the at least one wheel to rotate through the drive assembly; and
the handlebar assembly according to claim 1, wherein the handlebar assembly is connected to the bike frame through the support post.

10. The exercise bike of claim 9, further comprising a display and computing device connected to the handlebar assembly through a rotating component.

11. The exercise bike of to claim 10, wherein, the display and computing device is configured to play videos and audios; the exercise bike further comprises:
a plurality of bike sensor devices, wherein the bike sensor devices are configured to track and collect user performance data;
a control device configured to:
receive an exercise guiding video determined according to a selected music input/audio signal, wherein the exercise guiding video comprises a first exercise guiding video and/or a second exercise guiding video, the first exercise guiding video is a live video generated automatically according to the selected music input/audio signal, the second exercise guiding video is a video previously recorded according to the selected music input/audio signal;
receive CGA and special-effect/animated feedbacks;
control the display and computing device to display the exercise guiding video, the CGA, the special-effect/animated feedbacks and the selected music input/audio signal;
receive the user performance data from the bike sensor devices;
receive interactive feedback data generated or updated according to a result obtained by matching the user performance data with music information/audio signal analyzed from selected music input/audio signal;
control the display and computing device to display the interactive feedback data.

12. The exercise bike of claim 1, wherein, the exercise guiding video is generated by:
extracting the music information/audio signal from the selected music input/audio signal;
generating a movement instruction sequence automatically by matching and analyzing movements in a template exercise movement database/inventory, according to the music information/audio signal and a persona and user behavior pattern, or according to a user selection;
generating the exercise guiding video according to the movement instruction sequence.

13. The exercise bike of claim 12, wherein, the music information/audio signal of the selected music input/audio signal comprises music attributes/features and a timeseries/sequence with signals of rhythmic events/features, the movement instruction sequence is generated by:
matching and analyzing at least one movement instruction unit sequentially from a template exercise movement database/inventory, according to the music attributes/features and the timeseries/sequence with signals of rhythmic events/features, wherein the template exercise movement database/inventory includes a plurality of movement instruction units;
generating a movement instruction sequence according to a sequence of the movement instruction units.

14. The exercise bike of claim 13, wherein, the timeseries/sequence with signals of rhythmic events/features comprises a plurality of segments with signals of rhythmic events/features, the music attributes/features comprise a variety of measurements or quantification of music energy, the music attributes/features further comprise one or more of music duration, music segments, lyrics, genre, and artist;
wherein the step of matching and analyzing at least one movement instruction unit sequentially comprises:
obtaining an ith segment with signals of rhythmic events/features, and searching for at least one succeeding movement instruction unit option to a pre-defined (i−l)th movement instruction unit;
obtaining a pre-determined movement energy-transition probability distribution for transitioning the (i−l)th movement instruction unit to its succeeding movement instruction unit (the ith movement instruction unit) based on the movement energy level of the (i−l)th movement instruction unit and a model/mechanism of varying/transitioning movement energy levels from one to another;
dynamically updating/adjusting the pre-determined movement energy-transition probability distribution described above for transitioning the (i−l)th movement instruction unit to its succeeding movement instruction unit (the ith movement instruction unit) when variable measurements or quantification of music energy/audio signals and user performance data are received;
determining the energy level of the succeeding movement instruction unit option to the (i−l)th movement instruction unit based on the movement energy-transition probability distribution for transitioning the (i−l)th movement instruction unit to its succeeding movement instruction unit (the ith movement instruction unit);
selecting at least one succeeding movement instruction unit to the (i−l)th movement instruction unit as the ith movement instruction unit, according to the determined movement energy level of the (i−l)th movement instruction unit, or the persona and user behavior pattern;
wherein i is an integer ranging from 2 to N, and N is a number of the segment with signals of rhythmic events/features in the timeseries/sequence with signals of rhythmic events/features.

15. The exercise bike according to claim 12, wherein, the generated exercise guiding video is the first exercise guiding video, the exercise guiding video is generated by:
determining an instructor object and generating the first exercise guiding video according to the movement instruction sequence and the instructor object, wherein the instructor object is a virtual instructor or a real instructor; or, determining a virtual scene/stage or extended reality generated by CGA, and generating the first exercise guiding video according to the movement instruction sequence and the virtual scene/stage or extended reality generated by CGA, wherein the virtual scene/stage or extended reality generated by CGA has dynamically varying effects corresponding to the movement instruction sequence to improve engagement and immersiveness.

16. The exercise bike of claim 15, wherein, the instructor object is determined by matching and analyzing the virtual instructor or the real instructor according to the music information/audio signal of the selected music input/audio signal and/or the persona and user behavior pattern; or, determining the virtual instructor or the real instructor according to a user selection;
the virtual scene/stage or extended reality generated by CGA is determined by matching and analyzing the virtual scene/stage or extended reality generated by CGA according to the music information/audio signal of the selected music input/audio signal and/or the persona and user behavior pattern.

17. The exercise bike of claim 12, wherein, the generated exercise guiding video is the second exercise guiding video, the exercise guiding video is generated by:
generating a movement instruction/cuing list according to the movement instruction sequence;
playing the movement instruction/cuing list and the selected music input/audio signal;
receiving a recorded video as a pre-determined second exercise guiding video, wherein the pre-determined second exercise guiding video comprises a front layer including an instructor object and a recorded background, the recorded background is a green screen, and the instructor object of the front layer is a real instructor;
obtaining the second exercise guiding video by extracting the front layer including the instructor object from the pre-determined second exercise guiding video.

18. The exercise bike of claim 11, wherein, CGA and the special-effect/animated feedbacks are generated by:
matching, analyzing, integrating and synchronizing the CGA and special-effect/animated feedbacks according to the music information/audio signal of the selected music input/audio signal and/or the persona and user behavior pattern;
the step of playing the exercise guiding video, the CGA, the special-effect/animated feedbacks and the selected music input/audio signal further comprises:
updating and rendering the CGA and/or the special-effect/animated feedbacks according to the received user performance data.

19. The exercise bike of claim 11, wherein, the musical characteristics of the selected music input/audio signal comprises a timeseries/sequence with signals of rhythmic events/features, the timeseries/sequence with signals of rhythmic events/features comprises a plurality of segments with signals of rhythmic events/features;
wherein the step of displaying interactive feedback data comprises:
determining whether the user performance data coincides or synchronizes with the segment with signals of rhythmic events/features of a corresponding time;
if yes, displaying a combo-strike effect;
if no, displaying a special-effect/animated feedback showing “missing” or not displaying any special-effect/animated feedback on the display and computing device;
determining whether a user performance level should be raised or not according to a number of continuous displays of the combo-strike effect or a cumulative number of displays of the combo-strike effect;
if no, displaying a special-effect/animated feedback corresponding to no upgrading/leveling-up or not displaying any special-effect/animated feedback on the display and computing device;
if yes, displaying a special-effect/animated feedback corresponding to upgrading/leveling-up on the display and computing device, calculating a performance score of the user according to the user performance data, and displaying the performance score on the display and computing device.

20. The exercise bike of claim 11, wherein, the control device is further configured to:
establishing a virtual room or arena, and playing a same selected music input/audio signal, and the exercise guiding video, the CGA, the special-effect/animated feedbacks generated according to the same music input/audio signal on display and computing devices of the user and other users in the virtual room or arena;
receiving the user performance data;
calculating the performance score of the user according to the user performance data;
receiving the performance scores of the other users in the virtual room or arena;
displaying, in a leaderboard display area on the display and computing device, the performance scores calculated at a same timing and location of the selected music input/audio signal of the user and other users in the virtual room or arena in a sequence from large to small;
wherein, the displayed sequence of the performance scores is dynamically updated according to the variation of the performance scores.
